(19) United States
(12) Patent Application Publication
(10) Pub. No.: US 2019/0169665 A1
(43) Pub. Date: Jun. 6, 2019

(54) Gene Synthesis by Self-Assembly of Small Oligonucleotide Building Blocks

(71) Applicant: MLP HOLDING APS, Copenhagen (DK)

(72) Inventors: Morten Lorentz Pedersen, Cophenhagen NV (DK); Gitte Laurette Pedersen, Montauk, NY (US); Tanya Sharlene Kanigan, Charlotte, VT (US)

(21) Appl. No.: 16/023,960

(22) Filed: Jun. 29, 2018

Related U.S. Application Data
(63) Continuation of application No. 14/602,967, filed on Jan. 22, 2015, now abandoned.

(51) Int. Cl. C12P 19/34; C12N 15/10; C12N 15/66

(52) U.S. Cl. CPC C12P 19/34; C12N 15/1031; C12N 15/66; C12N 15/1027

(57) ABSTRACT

The invention provides a process for synthesizing genes and other long double stranded polynucleotides by assembling very short oligonucleotides into partly double stranded polynucleotides, and then connecting these partly double stranded polynucleotide subassemblies with linkers comprised of very short oligonucleotides. In one embodiment, the correct order of the polynucleotide subassemblies is coded in overhangs present at each end of the partly double stranded polynucleotide subassemblies. Linkers having a sequence complimentary to the combined overhangs connect adjacent subassemblies, which are then ligated together. In one preferred embodiment the oligos are six bases long, for which there are only 4096 different possible sequence permutations. A complete library of oligos of this size and scale can be cost-effectively synthesized and quality controlled, avoiding the typical errors and yield issues associated with phosphoramidite synthesis of longer oligos. Furthermore, the limited oligo library size supports development of a laboratory-scale gene synthesis machine.

### FIELD OF THE INVENTION

The present invention is in the technical field of synthetic biology. More particularly, the invention relates to systems and methods for polynucleotide synthesis and assembly and is applicable at all scales greater than a few base pairs, and preferably at scales equal to a hundred base pairs and higher.

### BACKGROUND OF THE INVENTION

State-of-the-art genome building relies upon inexpensive and massively parallel synthesis of single stranded oligonucleotides, as well as on the isolation of double stranded polynucleotides from nature. This field further relies upon purposeful assembly of these oligonucleotide and polynucleotide building blocks into longer double stranded polynucleotide constructs, including synthetic genes, through enzyme-aided processes that join polynucleotides together.

Despite many recent advances in the synthesis of DNA and other naturally occurring as well as artificial polynucleotides, this field is still limited by the cost and technical challenges associated with accurately producing polynucleotides, especially ones longer than a hundred bases. In vitro synthesis of polynucleotides is currently limited by the finite coupling efficiency of each nucleotide addition step. For example, the theoretical yield when coupling together 200 bases is less than 1% at 97.5% coupling efficiency and less than 0.1% at 96.5% coupling efficiency.

Furthermore, assembling together a large number of polynucleotides in a pre-specified order is technically difficult and remains prohibitively expensive for sequences greater than, say, 10 kilobase pairs. Consequently, to this day, although a mitochondrial genome (Gibson D G et al. 2010) has been synthesized and assembled entirely in vitro, the only published synthesis of a full prokaryotic genome was accomplished using a combination of in vivo and in vitro methods (Gibson D G et al, 2008). Until better tools and methods becomes available, researchers will continue to rely upon time consuming in vivo genetic engineering approaches and gene isolation methods for producing polynucleotides of significant length and complexity.

### PRIOR ART

Oligonucleotides are commonly synthesized on solid supports using sequential nucleotide coupling reactions based on phosphoramidite chemistry. This method is well established and multiple commercial manufacturers offer quick and inexpensive custom oligonucleotide synthesis. While it is theoretically possible to synthesize single stranded polynucleotides with more than 200 nucleotides (nt) through such single base addition reactions, yields decrease significantly with increasing polynucleotide length and this limits practical lengths to below 200 nt. As a consequence there is typically a significant surcharge for purchasing oligos longer than, for example, 80 bases.

Once synthesized, single stranded oligos can be assembled into double stranded polynucleotide sequences using methods described in the literature e.g., Stemmer et al. 1995; Smith et al. 2003; Xiong et al. 2004; Xiong et al., 2006; Gibson 2009. Most prominently, Polymerase Cycling Assembly (PCA) (Stemmer et al. 1995) uses a non-amplifying polymerase chain reaction (PCR) to link oligonucleotides together to form longer double stranded polynucleotide molecules up to approximately 3 kb in length. The oligonucleotides are typically in the range of 40 to 50 base pairs (bp) in length and are tiled together with ˜20 bp overlaps. A polymerase is then used to fill in gaps between oligos.

In vivo methods for assembling chemically synthesized oligonucleotides into genes and other polynucleotide sequences are also known in the art; e.g. Gibson, 2009. Gibson showed that yeast base assembly is suitable for assembling oligos up to 200 bp having overlaps of 20 nt or greater.

Overlapping oligonucleotides are more commonly assembled into double stranded polynucleotides in vitro using ligation chemistry. Parker et al. (US 2003/0228602) disclose successive ligation of oligonucleotide precursors of minimum 10 nt on a solid support to form a predefined polynucleotide sequence. As with yeast-based assembly, PCA and other oligonucleotide assembly methods known in the art, the preferred length of the oligo building blocks is in the range of 30-60 nt.

In contrast, Coope et al. (US 2001/0287490) disclose that complex DNA structures can be efficiently and accurately assembled by annealing and ligating very short oligonucleotides onto a partly double stranded dsDNA molecule attached to a solid support. Using this approach, the inventors demonstrated assembly of a 128 bp gene segment from a set of 8-mers using T4 DNA ligase (Horspool et al. 2010).

Another example of assembly of very short oligonucleotides is provided by Dunn et al. (1995), who demonstrated that single-stranded oligonucleotide primers between 12 and 30 nt long could be produced from a library of hexamer precursors in solution. Small sets of phopsphorylated oligonucleotide hexamers were first aligned in a predetermined order onto a scaffold of overlapping non-phosphorylated hexamers then ligated together using T4 or T7 ligase (Dunn et al, 1995). Afterwards the non-phosphorylated hexamers were removed from the single-stranded ligation product.

Once assembled from oligonucleotides, synthetic polynucleotides in some cases comprise the final product, and in other cases they comprise polynucleotide subassemblies to be linked together into larger constructs, including genes and gene cassettes. Polymerase Cycling Assembly may be used for this purpose (Smith et al. 2003); however, a newer gene assembly method, called Gibson Assembly (Gibson et al. 2009) is most commonly used to connect multiple double stranded polynucleotides into larger constructs.

Gibson Assembly efficiently joins multiple double stranded polynucleotides (10 to 20) with overlapping sequence homology in a single-tube isothermal reaction using three enzymes: T5 exonuclease, Phusion DNA polymerase and Taq ligase. The end product can be a linear double stranded DNA molecule, or a circularized double stranded DNA. Overlapping regions can be added to blunt ended DNA by using PCR with primers that contain adapter sequences. Thus Gibson Assembly can be used to join together blunt ended double stranded DNA polynucleotides. This method provides ease-of-use, flexibility and ability to produce large DNA construct; and has therefore been rapidly adopted by the synthetic biology community. Practitioners have assembled diverse products including oligonucleotides, DNA with varied overlaps (15-80 bp) and polynucleotides hundreds of kilobases long.

In both Gibson Assembly and Polymerase Cycling Assembly overlapping oligonucleotides at the ends of the building blocks must be present. Other polynucleotide assembly methods, including BglBrick Assembly (Anderson J C et al. 2010), use type II restriction endonucleases to create single stranded overhangs in double stranded DNA strands, and then they use ligase to join polynucleotides together after complimentary overhangs have been annealed. Such methods have the disadvantage of requiring the presence of appropriate enzyme restriction/recognition sites in all double stranded polynucleotides to be assembled into larger polynucleotides. Practioners of BglBrick Assembly circumvent this requirement by creating double stranded DNA subassemblies that are comprised of functional coding sequences with flanking restriction sites outside of coding regions.

Yet another polynucleotide assembly method, Golden Gate Assembly (Engler C. et al. 2008), makes use of Type IIS restriction endonucleases to create short overhangs in double stranded DNA, that are outside of the recognition site. The enzyme recognition sites can be added onto the polynucleotides in a PCR reaction, and thus an overhang can be created at will to produce complementary overhangs. The overlapping complimentary overhangs anneal together and are then joined by ligation. The Golden Gate process is sequence-independent and permits assembly of repeats with identical or highly homologous sequences, since only short (typically 4 bp) fusion sites at the end of the repeats have to be unique. An important caveat of this method is that the enzyme recognition site must be absent from the internal sequences of all DNA segments.

Overhangs in double stranded DNA can also be created without use of restriction endonucleases. U.S. Pat. No. 6,358,712 describes methods for producing overhangs in DNA molecules through a PCR based method. This approach to creating overhangs provides a means for building double stranded polynucleotides by joining together shorter double stranded polynucleotides with complementary overhangs using ligation chemistry. This method, like the Golden Gate process relies upon the availability of suitable polynucleotides to serve as building blocks for a larger polynucleotide construct, and thus does not provide means for de novo synthesis of an artificial gene or other large polynucleotide constructs.

Another key limitation of current polynucleotide synthesis and assembly methods derives from errors that occur during synthesis of nucleic acid building blocks and in coupling of building blocks together. These errors accumulate and are thus a function of the final product length. PCR amplification steps, if included, introduce additional sequence errors. Microarray based syntheses are also known to have even higher error rates (Ma S et al. 2012). Correction methods, such as use of mismatch cleaving endonuclease (Quan J et al. 2011), and other methods are employed to increase the accuracy of microarray gene synthesis; however, error rates for high throughput synthesis methods are still unacceptably high for many industrial applications.

Recently, a new approach to de novo synthesis of oligo and poly-nucleotides, including long polynucleotides, has been reduced to practice by Gen9, Incorporated. U.S. Pat. No. 8,058,004 teaches production of mixtures of long, gene-length polynucleotides through assembly of multiple shorter oligonucleotides that are synthesized in situ on a microarray platform. A series of repeated cycles of primer extension on the array surface is followed by release of the resulting library of polynucleotides into solution using restriction endonucleases. Although this combinatorial method is well suited for creating a library of diverse sequences for screening and optimization experiments, it is not an efficient method for purposeful assembly of a single, large DNA construct of predefined sequence. Thus, although proven to be automatable, current microarray based gene synthesis methods are not enabling for a universal gene synthesizer; a machine that could synthesize single pre-specified genes and other long DNA sequences of arbitrary sequence at prices and delivery times competitive with industrial gene suppliers such as IDT and Blue Heron Biotechnology.

Furthermore, none of the gene synthesis methods known in the art provides a coherent scalable solution for construction of pre-specified polynucleotide sequences of gene length or longer from oligonucleotide building blocks less than 10 nt long. As such these methods cannot take into account the significant redundancy of nucleotide sequences present in the genomes of all living beings. As a result, practitioners of the current art experience, at best, a linear relationship between the size/complexity of the genome and the cost of synthesizing it.

### SUMMARY OF THE INVENTION

The present invention provides a process for in vitro synthesis and assembly of double stranded polynucleotides, through self-assembly of multiple short single stranded oligonucleotide building blocks. The present invention further provides an improved system for assembly of hundreds to hundreds of millions of double stranded polynucleotides into larger polynucleotide constructs, including gene-length constructs, whole chromosomes, and elaborate gene cassettes, using short single stranded oligonucleotides as linkers to connect pairs of double stranded polynucleotides.

In particular, the present invention provides a process for synthesizing genes and other long double stranded polynucleotides by assembling together very short oligonucleotides in solution into polynucleotide subassemblies, and then connecting these subassemblies with linkers comprised of very short oligonucleotides. In one preferred embodiment the oligos are six bases long, for which there are only 4096 different possible sequence permutations. A complete library of oligos of this size and scale can be cost-effectively 1) synthesized using standard phosphoramidite chemistry, 2) purified, and 3) quality controlled, avoiding the typical errors and yield issues associated with phosphoramidite synthesis of longer oligos. Furthermore, the limited oligo library size supports development of automated processes. Thus the present invention enables development of a gene synthesis machine; one that can produce ANY possible sequence of a polynucleotide, including whole genomes, from standardized building blocks (e.g. all the 4096 permutations of single stranded hexamers).

In one preferred embodiment of this invention, the double stranded polynucleotide assembled from single stranded oligonucleotides comprises the final product and can be purified and copied using PCR, clonal selection and other techniques well known in the art. In another preferred embodiment of this invention, the newly assembled double stranded polynucleotide molecule comprises a subassembly that can be then linked to other subassemblies to create larger polynucleotide constructs. In this embodiment, the correct order of the subassemblies is coded in overhangs at both ends of the subassembly molecules. Linkers having a sequence complimentary to the combined overhangs connect adjacent subassemblies in the final construct and the ligation is performed under high-fidelity conditions that block side reactions and minimize mismatches. The preferred length for these overhangs are three bases, and a six base oligonucleotide linker is used to connect two adjacent polynucleotides that comprise a 3′ overhang on one molecule and a 5′ overhang on the other molecule, respectively; however, it is possible to obtain stringent ligation with overhangs several bases longer, and possibly up to seven bases long or longer, by optimizing the ligation reaction.

For genes and other long polynucleotide targets, software may be developed to select optimum synthesis strategy. Taking advantage of the sequence redundancy present in all genomes this approach effectively breaks the linear relationship between the size/complexity of a genome and the cost of synthesizing it. This, in turn, enables the synthesis of significantly more complex genomes in similar time and with similar cost to that currently required to synthesize much smaller genomes.

In another preferred embodiment of the present invention, a method for shuffling segments of sequence within a larger DNA construct, including so-called “exon shuffling,” is provided. A set of polynucleotides is connected in multiple orders to produce multiple different product molecules in a single ligation reaction. For this application the appropriate oligonucleotide linkers are included in one or a series of different assembly reactions to connect at least two polynucleotides together in two different orders. For example, Polynucleotide A having overhang ZIP1 is connected to both Polynucleotide B with overhang ZIP2, and Polynucleotide C having overhang ZIP3, by linkers ZIP1-ZIP2 and ZIP1-ZIP3.

A feature of this method is that pre-knowledge of the full sequence that is to be modified is not required; only short stretches of sequence between the regions (e.g. exons or genes) to be shuffled must be known to the person practicing the method. Thus, for example, the practitioner could order the linker oligonucleotides from a supplier without revealing the sequence of a proprietary gene.

In yet another preferred embodiment, a library of diverse double stranded polynucleotide constructs is assembled from libraries of single and/or double stranded polynucleotide building blocks. In the present invention, double stranded polynucleotide libraries can be used as building blocks, whereas single stranded oligonucleotide libraries can be used both as oligonucleotide building blocks and as linkers. Both types of libraries can be prepared using methods known in the art, including methods involving isolation from biological sources and methods involving do novo synthesis.

In summary, methods are provided by this invention for decreasing cost and increasing accuracy of synthesizing large polynucleotides, for gene shuffling and for other approaches to engineer sequence diversity. Together these methods provide a rich toolset for gene optimization. Through the present invention, entire systems of genes can be optimized to increase the productivity of biological systems in industrial biotechnology; including biofuel and waste disposal, as well as the production of therapeutic proteins and other complex biologically derived chemicals.

### BRIEF DESCRIPTION OF THE DRAWINGS

FIG. 1 depicts parallel assembly of three oligonucleotides.

FIG. 2 depicts assembly of two partly double stranded polynucleotides by connecting either two 3′ overhangs or two 5′ overhangs on two separate polynucleotides.

FIG. 3 depicts assembly of two partly double stranded polynucleotides using an oligonucleotide linker to connect one 3′ overhang and one 5′ overhang on two separate polynucleotides.

FIG. 4 depicts parallel assembly of two oligonucleotides onto a partly double stranded seed.

FIG. 5 depicts sequential assembly of oligos onto a seed in combination with a partly double stranded cap molecule.

FIG. 6 depicts assembly of multiple partly double stranded polynucleotides and multiple oligonucleotide linkers derived from multiple processes.

FIG. 7 depicts a simple gene shuffling application.

FIG. 8 contains a flowchart describing the algorithm for determining which oligonucleotides can be assembled together to form sets of subassemblies that can be linked together in only one order (i.e., the subassemblies form a non-ambiguous assembly) for the purpose of synthesizing a particular gene sequence.

FIG. 9 depicts processes described in the flowchart of FIG. 8 being applied to a particular DNA sequence.

### GLOSSARY

‘Building blocks’ shall refer to nucleotides that can be assembled to larger molecules, which can be either final products or building blocks themselves.

‘Cap’ shall refer to a partly double stranded polynucleotide molecule having only one single stranded overhang at one end comprising 1 or more bases; this molecule may function as a ‘cap’ in an assembly of multiple oligo-/polynucleotide building block in terms of comprising the last polynucleotide building block added to the assembly. A ‘cap’ always comprises only one nucleic acid zip code as its overhang. A ‘cap’ may also comprise one or more functional sequences within its double stranded part including, but not limited to: a spacer sequence and a biotin linker to link the seed to a magnetic bead; a release site (see definition below); a PCR primer site; a label; and/or a polynucleotide sequence that will be part of the final product.

‘Nucleic acid zip codes’ or ‘zip code’ shall refer to a unique short single stranded nucleic acid sequence that is complementary to another zip’ code, and thereby are used to direct assembly of oligo-/polynucleotide building blocks in a particular order through a complimentary overlapping sequence.

‘Oligonucleotides’ and ‘oligos’ shall refer to single stranded nucleic acids that are generally shorter than 50, 100, 150 or 200 bases in length. Commonly made in the laboratory by solid-phase chemical synthesis, these small bits of nucleic acids can be manufactured with any user-specified sequence.

‘Overhang’ shall refer to the part of partly double stranded oligo-/polynucleotides that is single stranded.

‘Polynucleotides’ shall refer to single or double stranded nucleic acids that are generally longer than 50, 100, 150, or 200 bases in length.

‘Release site’ shall refer to a chemical feature within a polynucleotide seed or cap molecule that enables the final product to be released from the seed or cap. The release site can be, for example, a recognition site for a restriction/nicking endonuclease, or one or more uracil residues.

‘Seed’ shall refer to a partly double stranded polynucleotide molecule having only one single stranded overhang at one end comprising 1 or more bases; this molecule may function as a ‘seed’ in an assembly of multiple oligo-/polynucleotide building blocks in terms of comprising the first polynucleotide building block added to the assembly. A ‘seed’ always comprises one nucleic acid zip code as its overhang. A ‘seed’ may also comprise one or more functional sequences within its double stranded part including, but not limited to: a spacer sequence and a biotin linker to link the seed to a magnetic bead; a release site (see definition below); a PCR primer site; a label; and/or a polynucleotide sequence that will be part of the final product.

‘Single stranded tag’ shall refer to consecutive nucleotides linked together and forming a single stranded oligonucleotide. The number of nucleotides may range typically from about 2 to 20 but can also be more than 20 nucleotides, including tags of more than e.g. 200 nucleotides. For the purposes of this patent, a single stranded nucleotide tag can be obtained from genetic material present in a biological sample and can also be obtained from synthetic oligonucleotides.

‘Subassembly’ shall refer to a nucleic acid molecule assembled from a set of oligonucleotide building blocks.

‘Tag library’ shall refer to a plurality of at least one single stranded tag.

‘Wobble zip’ shall refer to part of the zip code sequence that contains all possible permutations of such sequence code or a subset of all possible permutations of such sequence code.

### DETAILED DESCRIPTION OF THE INVENTION

The following descriptions relate to preferred embodiments of the invention and involve assembling large, even gene-length, double stranded polynucleotides using single stranded oligonucleotides of preferably six bases (i.e. hexamers) together with partly double stranded polynucleotide molecules having three base overhangs; however, the preferred embodiments of the invention are not limited to any one length of overhang and single stranded oligonucleotides having lengths up to more than 20 bases and overhangs up to more than 10 bases can be applied.

In one preferred embodiment of the invention, the oligonucleotides are all six bases long and the overhangs are three nucleotides long. The oligonucleotides are used to connect the double stranded polynucleotides to one another and to the seed through complimentary sets of nucleotide bases; here referred to as molecular zip codes. Each 3-nucleotide sequence provides one of 64 (4³) possible molecular zip codes; whereas the use of a six-nucleotide linker provides for up to 4096 (48) different polynucleotide pairings. Larger numbers of pairings are possible with longer oligo linkers and complementary overhangs.

The invention enables more than one building block at the same time because the correct order of assembly is coded into the overhangs. This simplifies the polynucleotide manufacturing process and dramatically increases the synthesis speed because all possible permutations of the single stranded oligonucleotides can be pre-ordered. As such, this invention supports development of a whole gene synthesizing machine that can produce ANY possible sequence of a polynucleotide from a limited set of standardized building blocks (e.g. all the 4096 permutations of single stranded hexamers).

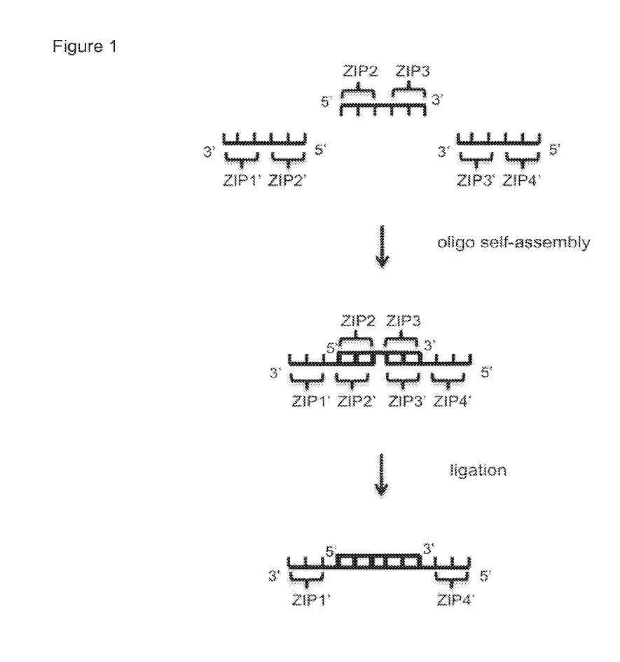

FIGS. 1 to 3 illustrate three types of oligonucleotide assembly reactions used in one preferred embodiment of this invention.

FIG. 1 depicts the assembly of three hexamers into a double-stranded polynucleotide having one 3′ and one 5′ overhang. In one preferred embodiment the oligonucleotides can only be assembled in the order specified by their consecutive overlapping bases; here referred to as a nucleic acid ‘zip code’. In one preferred embodiment after the oligos anneal together in solution, a phosphodiester bond is formed between adjacent oligos using a ligation reaction to create a continuous strand hybridized to its complementary strand. Suitable conditions for ligation must be established to ensure that only oligos that exactly match the single stranded overhang available for hybridization are added to the growing chain. Ligation conditions would comprise e.g. choice of ligase, buffer composition, reaction temperature, and be chosen and optimized using methods known in the art. The product of this simple assembly is a partly double stranded polynucleotide having one 3′ overhang and one 5′ overhang on the lower strand. A similar process can be applied to create a partly double stranded polynucleotide having one 3′ overhang and one 5′ overhang on the upper strand.

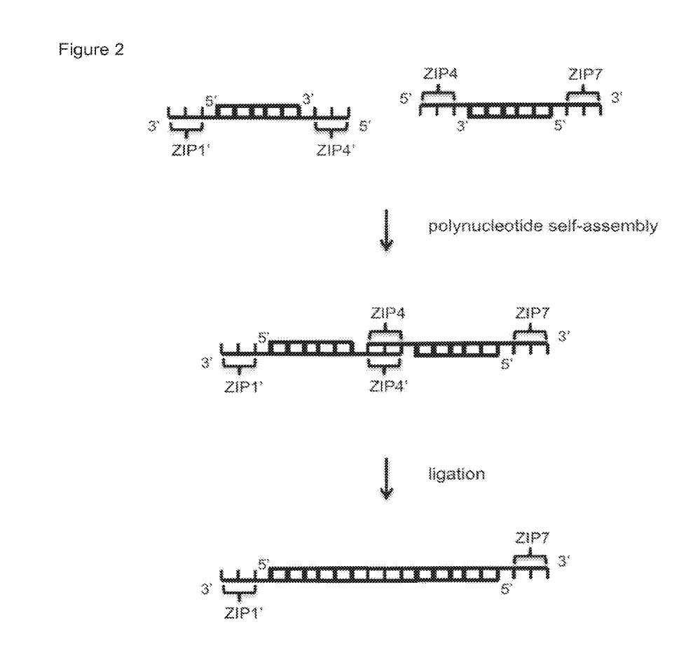

In FIG. 2 two partly double stranded polynucleotides derived from the assembly of oligonucleotides depicted in FIG. 1 are assembled together through the complementary zip codes that comprise their 3′ overhangs. After ligation creates phosphodiester bonds between strands, the result is a larger, partly double stranded, polynucleotide. This molecule may be the intended end product, or it may serve as a building block for further assembly reactions.

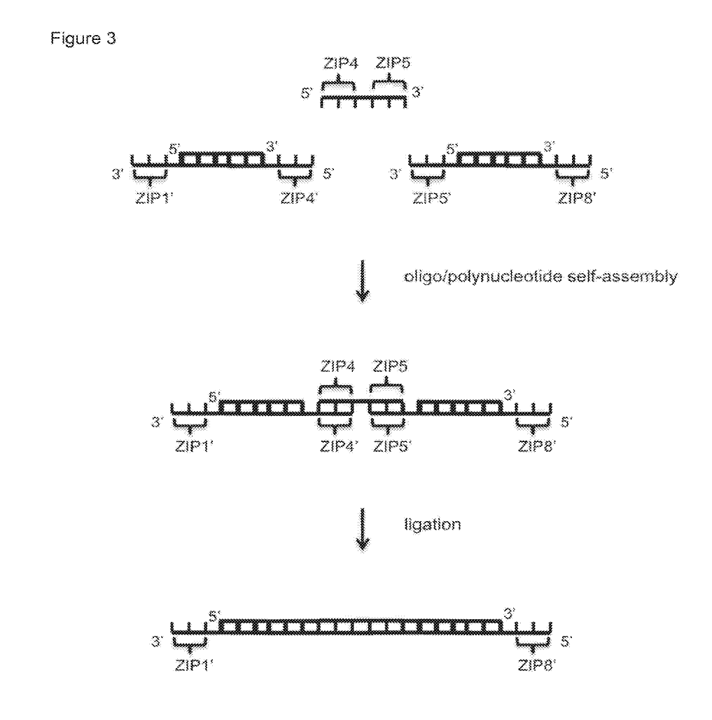

Alternatively, partly double stranded polynucleotides can be connected together in a particular order using single stranded oligonucleotide “linkers” to bridge adjacent overhangs. FIG. 3 shows how a single stranded hexamer linker connects the 5′ overhang on the lower strand from a first polynucleotide with a 3′ overhang on the lower strand from a second polynucleotide. After the molecules anneal together they can be ligated to form the new larger double stranded polynucleotide.

The product of the assembly, which may comprise one or more subassemblies or one or more final constructs, may be isolated from the reaction by PCR, clonal selection and other methods well known in the art. Under certain conditions, such as those in which ligation is not strict or when ambiguous linkers are present (e.g. pallindromes), side products may be produced. These unintended polynucleotides are unlikely to have the same length as the desired product. Thus size selection, e.g. using gel electrophoresis, may be an additional means of isolating the desired product from these side-products, if any.

Another means of separating the intended product and side product(s) is by selective capture of the overhangs. Alternate assemblies of a given set of oligos and/or partially double stranded polynucleotides are unlikely to possess the same sets of overhangs. Thus the product can be isolated by (1) capturing the intended product on a surface-bound capture molecule having a three base overhang—or simply three bases of single stranded DNA on a spacer attached to a surface—complimentary to the first overhang on the intended product, then (2) capturing the intended product on a surface-bound polynucleotide having a three complimentary bases available for capture to the second overhang on the intended product and (3) releasing products that are captured by steps 1 and 2 into solution by methods known in the art. It may or may not be desirable to release the polynucleotides in step 1 before proceeding to step 2. For example the intended product, if sufficiently long, can be captured on a surface or matrix displaying capture sequences complimentary to both overhangs. Nucleotide analogs and/or ligation can be used to increase the efficiency and stringency of the capture conditions and followed by release of the product (or subassembly) from the surface or matrix using methods described in this application or otherwise known in the art.

Oligos and/or polynucleotides can also assemble on a partially double stranded polynucleotide that has only one overhang. We shall refer to such a molecule as a ‘seed’ when its overhang comprises the first zip code for a growing assembly. In one embodiment of the invention, the seed is comprised of a partly double stranded polynucleotide spacer molecule having a single stranded 3-base overhang (ZIP1′) at one end. This molecule can be bound to the surface of a solid support such as a paramagnetic bead at its double stranded end; such that the single stranded portion is free to bind with any purely single stranded or single stranded part of a partly double stranded oligo/poly-nucleotide molecule in solution having a complimentary 3-base sequence (ZIP1). The double stranded portion of this seed may contain a release site, such as a recognition/restriction site for a restriction/nicking endonuclease, or it may contain uracil residues; either of which can be used for release of the double stranded polynucleotide product from the solid support. This double stranded polynucleotide sequence may, optionally, include a PCR primer-binding site to be used to amplify the product sequence.

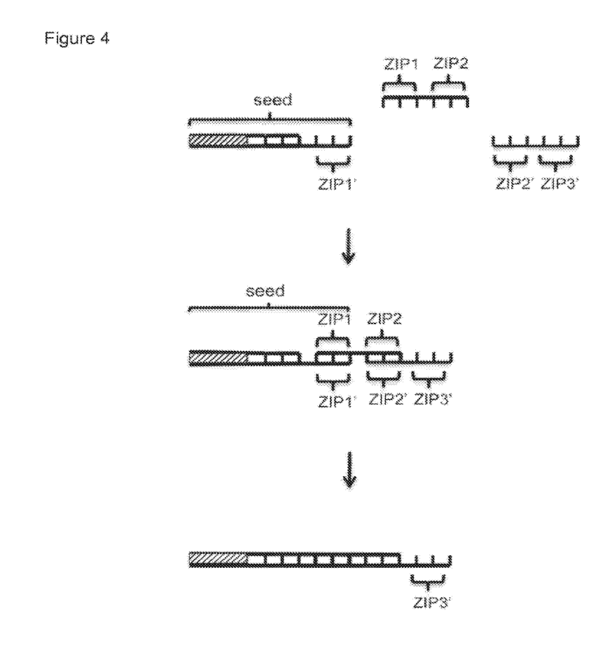

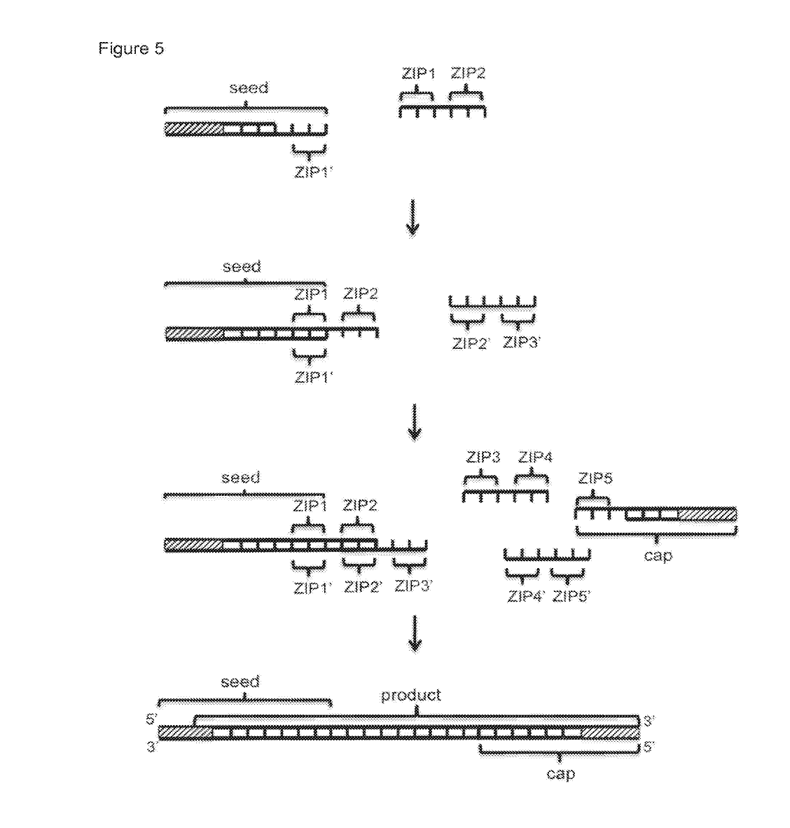

FIGS. 4 and 5 depict two embodiments of the polynucleotide assembly process wherein multiple overlapping oligonucleotides self-assemble on a seed to create a double stranded polynucleotide. In one preferred embodiment the oligonucleotide building blocks are all present together in a single pot mixture and self-assemble onto the seed in a parallel fashion, and are then subsequently ligated together (FIG. 4). In a separate preferred embodiment, subsets of oligos are added to the reaction mixture one-at-a-time in a step-wise fashion (FIG. 5). Also depicted in FIG. 5 is the inclusion of a ‘cap’ polynucleotide as a building block that can terminate a growing oligonucleotide chain because it does not provide a second overhang for additional assembly. A single stranded oligonucleotide can, alternatively, terminate a polynucleotide assembly if one of the two zip codes does not complement any other zip code present in the reaction mixture.

In these drawings the assemblies are depicted with the minimum number of oligos and polynucleotides to illustrate the concept; however, much larger numbers of oligonucleotides and/or polynucleotides can be assembled using methods enabled by this invention. Furthermore, these methods can be used to assemble oligonucleotides and polynucleotides derived from different biological sources and synthesized by different methods known in the art. In one preferred embodiment the partly double stranded polynucleotides are synthesized by means of the oligonucleotide self-assembly process described in this invention. In another preferred embodiment these polynucleotides are isolated from double stranded DNA derived from a biological source using restriction endonucleases and other cleavage agents known in the art. In particular, U.S. Pat. No. 6,958,217 teaches that single stranded oligonucleotide tags of fixed uniform length can be isolated from biological samples using the combined action of Type IIS restriction and nicking enzymes. This patent also provides a means for creating a library of polynucleotides having fixed length overhangs, which are the byproducts of the tag isolation process.

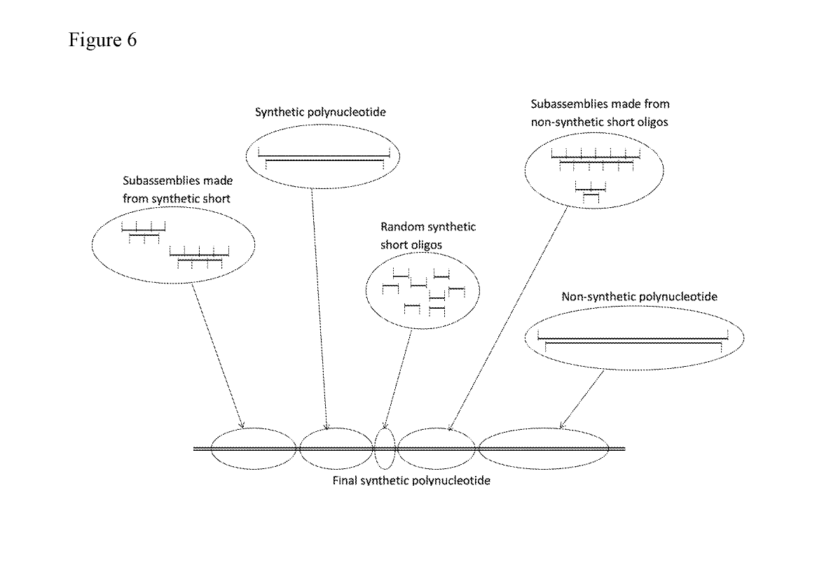

FIG. 6 illustrates the versatility of the method enabled by this invention by depicting a double stranded polynucleotide sequence assembled from building blocks that derive from a variety of sources and processes. These include synthetic and non-synthetic polynucleotides; subassemblies of synthetic and non-synthetic oligonucleotides, as well as random permutations of synthetic oligos. All of the building blocks have single stranded overhangs that can be connected directly (as shown in FIG. 2) or through an oligonucleotide linker (as shown in FIG. 3).

These overhangs and oligonucleotide linkers, which together comprise the zip codes, determine the desired order of the oligo and polynucleotides building blocks. In one preferred embodiment all of the zip codes are unique such that the polynucleotides can be assembled in a single pre-determined order to form a single product. In another embodiment one or more zip codes are repeated and/or degenerated such that the polynucleotides are combined in at least two ways to purposefully synthesize at least two distinct polynucleotide products (i.e., for gene shuffling and codon optimization applications).

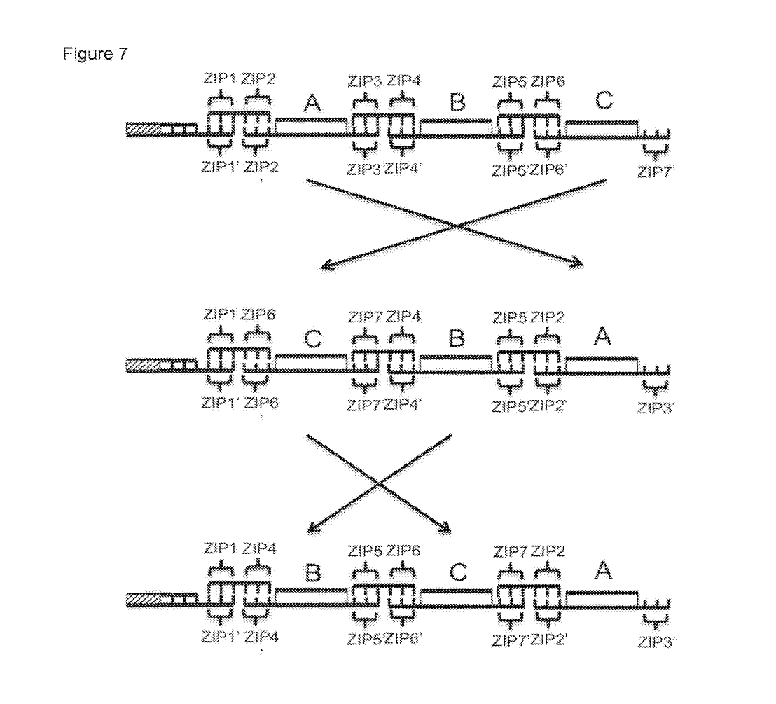

FIG. 7 contains a representation of a simple gene shuffling application. Three polynucleotide sequences are shuffled between three positions by including alternative oligonucleotides linkers in the reaction. The figure depicts three possible products, shown as surface-bound assemblies prior to ligation. The assembly at the top is comprised of the seed displaying overhang ZIP1′; three double-stranded polynucleotides (A, B, and C) each having two 3-base overhangs on the lower strand; and three oligonucleotide linkers (ZIP1-ZIP2, ZIP3-ZIP4 and ZIP5-ZIP6). These components assemble into the unique structure by virtue of their overlapping complimentary sequences. Two alternate polynucleotide sequences are created by including additional oligos (ZIP1-ZIP6, ZIP7-ZIP4, ZIP5-ZIP2, ZIP1-ZIP4, ZIP7-ZIP2) in the reaction mixture. A given set of olio/poly-nucleotide building blocks can also be shuffled by including at least one linker for which one of the two zip codes has been replaced by a wobble zip that can join one specific building block to any other building block (for example, ZIP5-NNN where N=A, C, G, or T).

Another embodiment of the present invention provides a means for introducing a frameshift into the synthesized gene. In this embodiment the oligonucleotide linker is at least one base longer than the combined length of its two zip codes. The extra base or bases create a gap in the other strand of the resulting oligo/polynucleotide assembly that can subsequently be closed by e.g. a DNA polymerase.

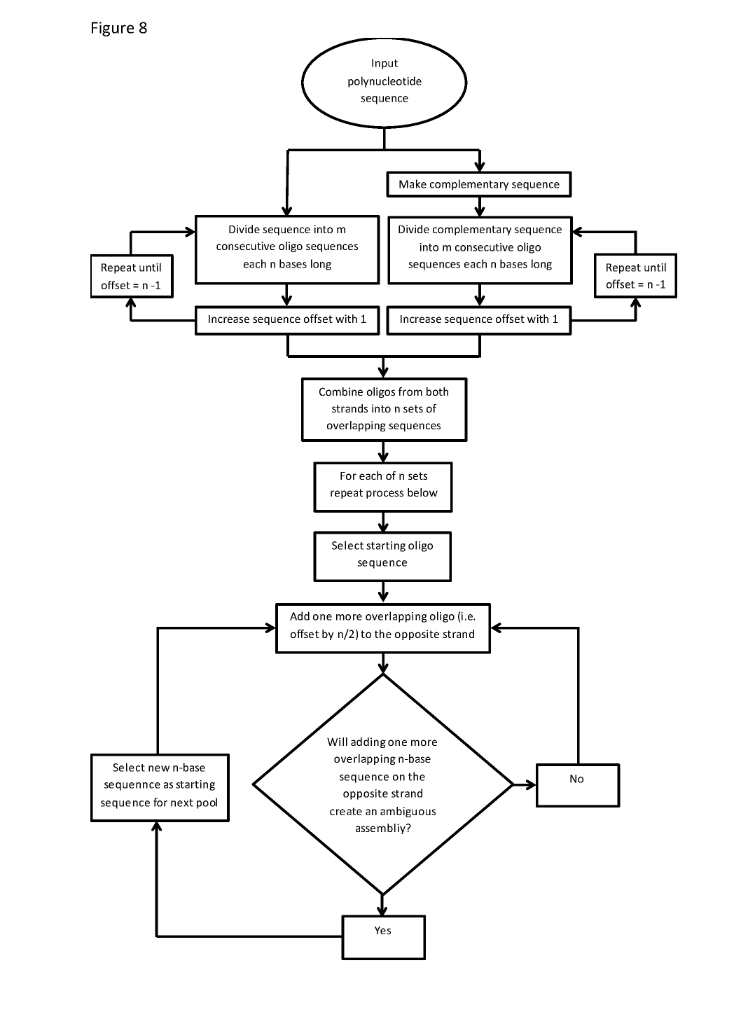

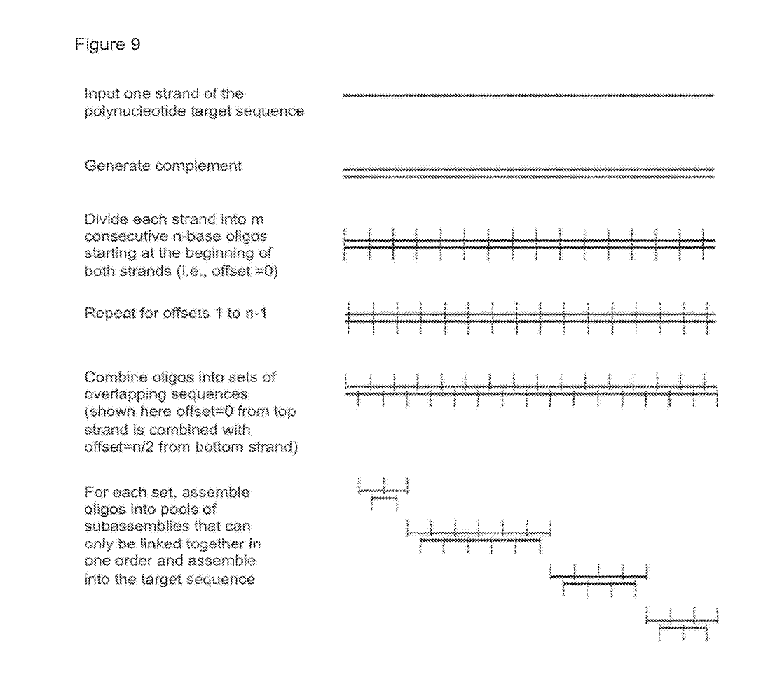

The invention also enables genes and other large polynucleotides to be synthesized by dividing the gene sequence into subassemblies comprised of pools of overlapping hexamers. If each pool of hexamers is chosen such that it can only be assembled in a single configuration (i.e., it forms an unambiguous assembly), side reactions can be minimized or eliminated; whereas combining all hexamer pools together in a single assembly process would result in multiple products. The resulting subassemblies are subsequently ligated together using their three-base overhangs in combination with connecting oligo hexamers to form the final product. This strategy enables multiple starting points for the synthesis of the gene and it is compatible with use of laboratory robotics. A flowchart showing a process for selecting pools of short oligonucleotide building blocks of e.g. six bases is depicted in FIG. 8. Accompanying this flowchart is a figure (FIG. 9) depicting the different in silico operations taking place on the target sequence.

A similar strategy is also possible with building blocks longer or shorter than six bases and it is very easy to automate. However, building blocks of six bases are preferred because they are long enough to create a three-base overhang suitable for ligation and yet also short enough to pre-order all sequence permutations. Furthermore, six is an even number that permits creation of overhangs having a uniform number of bases.

### REFERENCES

Anderson J C, Dueber J E, Leguia M, Wu G C, Goler J A, Arkin A P, Keasling J D. (2010) BglBricks: A flexible standard for biological part assembly, Journal of Biological Engineering, 4(1):1-12.
Dunn J J, Butler-Loffredo L L, Studier F W. (1995) Ligation of hexamers on hexamer templates to produce primers for cycle sequencing or the polymerase chain reaction. Anal Biochem. 228(1):91-100.
Engler C, Kandzia R, Marillonnet S. (2008) A one pot, one step, precision cloning method with high throughput capability. PloS One, 3(11):e3647.
Gibson D G, Benders G A, Andrews-Pfannkoch C, Denisova E A, Baden-Tillson H, Zaveri J, Stockwell T B, Brownley A, Thomas D W, Algire M A, Merryman C, Young L, Noskov V N, Glas s J I, Venter J C, Hutchison III C A, Smith H A. (2008) Complete Chemical Synthesis, Assembly, and Cloning of a Mycoplasma genitalium Genome. Science, 319(5867):1215-1220.
Gibson D G. (2009) Synthesis of DNA fragments in yeast by one-step assembly of overlapping oligonucleotides. Nucleic Acids Research, 37(20):6984-6990.
Gibson D G, Young L, Chuang R Y, Venter J C, Hutchison C A 3rd, Smith H O. (2009) Enzymatic assembly of DNA molecules up to several hundred kilobases. Nature Methods, 6(5):343-345.
Gibson D G, Smith H O, Hutchison C A, Venter J C, Merryman C. (2010) Chemical synthesis of the mouse mitochondrial genome. Nat Methods 2010a(7):901-905.
Hebelstrup K H, Christiansen M W, Carciofi M, Tauris B, Brinch-Pedersen H, Holm P B. (2010) UCE: A uracil excision (USER™)-based toolbox for transformation of cereals. Plant Methods, 6:15-24.
Horspool D R, Coope R J N, Holt R A (2010) Efficient assembly of very short oligonucleotides using T4 DNA Ligase. BMC Res Notes, 3:291-299.
Ma S, Saaem I, Tian J. (2012) Error correction in gene synthesis technology. Trends Biotechnol., 30(3):147-54.
Quan J, Saaem I, Tang N, Ma S, Negre N, Hui G (2011) Parallel on-chip gene synthesis and application to optimization of protein expression Nature Biotechnology. 29: 449-452.
Smith H O, Hutchison III C A, Pfannkoch C, and Venter J C (2003) Generating a synthetic genome by whole genome assembly: X174 bacteriophage from synthetic oligonucleotides. PNAS, 100(26): 15440-15445.
Stemmer W P, Crameri A, Ha K D, Brennan T M, Heyneker H L (1995) Single-step assembly of a gene and entire plasmid from large numbers of oligodeoxyribonucleotides. Gene, 1614: 49-53.
Xiong A S, Yao Q H, Peng R H, Li X, Fan H Q, Cheng Z M, Li Y. (2004) A simple, rapid, high-fidelity and cost-effective PCR-based two-step DNA synthesis method for long gene sequences. Nucleic Acids Res, 32(12):e98.
Xiong A S, Yao Q H, Peng R H, Duan H, Li X, Fan H Q, Cheng Z M, Li Y. (2006) PCR-based accurate synthesis of long DNA sequences. Nat Protoc, 1(2):791-797.
Xiong A S, Peng R H, Zhuang J, Liu J G, Gao F, Chen J M, Cheng Z M, Yao Q H. (2008) Non-polymerase-cycling-assembly-based chemical gene synthesis: strategies, methods, and progress. Biotechnol Adv. 26(2):121-34.

What is claimed is:

1. A method for synthesizing a double stranded polynucleotide molecule having a predefined sequence, the method comprising the steps of:
i) providing at least three single stranded oligonucleotides comprising complementary nucleotide sequence parts,
ii) contacting the least three single stranded oligonucleotides provided in step i) with each other, and
iii) creating at least one phosphodiester bond between any adjacent nucleotide in the self-assembled set of single stranded oligonucleotides from step ii) to create a double stranded polynucleotide of higher molecular weight than each of the individual single stranded oligonucleotides provided in step i).

2. Method of claim 1 comprising the further step of:
i) providing at least two double stranded polynucleotide molecules having a predefined sequence produced using the steps i) through iii) of claim 1,
ii) contacting the at least two double stranded polynucleotides provided in step i) with each other, and
iii) creating at least one phosphodiester bond between any adjacent nucleotide in the self-assembled set of double stranded polynucleotides from step ii) to create a double stranded polynucleotide of higher molecular weight than each of the individual double stranded oligonucleotides provided in step i).

3. Method of claim 1 comprising the further step of:
i) providing at least two double stranded polynucleotide molecules having a predefined sequence produced using the steps i) through iii) of claim 1,
ii) providing at least one single stranded oligonucleotide comprising complementary nucleotide sequence parts to overhangs at the ends of the at least two double stranded polynucleotide molecules provided in step i),
iii) contacting the at least two double stranded polynucleotides provided in step i) with the at least one single stranded oligonucleotide provided in step ii), and
iv) creating at least one phosphodiester bond between any adjacent nucleotide in the self-assembled set of double stranded polynucleotides from step iii) to create a double stranded polynucleotide of higher molecular weight than each of the individual double stranded oligonucleotides provided in step i).

4. A method for synthesizing a double stranded polynucleotide molecule having a predefined sequence, the method comprising the steps of:
i) providing at least two double stranded polynucleotide molecules having a predefined sequence,
ii) providing at least one single stranded oligonucleotide comprising complementary nucleotide sequence parts to overhangs at the ends of the at least two double stranded polynucleotide molecules provided in step i),
iii) contacting the at least two double stranded polynucleotides provided in step i) with the at least one single stranded oligonucleotide provided in step ii), and
iv) creating at least one phosphodiester bond between any adjacent nucleotide in the self-assembled set of double stranded polynucleotides from step iii) to create a double stranded polynucleotide of higher molecular weight than each of the individual double stranded oligonucleotides provided in step i).

5. Method of claim 1 to 4 wherein the creation of at least one phosphodiester bond is catalyzed by a ligase enzyme.

6. Method of claim 1 to 4 wherein the creation of at least one phosphodiester bond is substituted by combining, in a polymerase chain reaction, the individual oligonucleotides and polynucleotides into at least one double stranded polynucleotide of higher molecular weight than the each of the individual oligonucleotides/polynucleotides that went into the reaction.

7. Method of claim 2 to 4 wherein each of the at least two double stranded polynucleotide molecules provided in step i) comprises no more than one 3′ overhang and no more than one 5′ overhang.

8. Method of claim 2 to 4 wherein at least one of the at least two double stranded polynucleotide molecules provided in step i) is treated with a phosphotase prior to step iii).

9. Method of claim 2 to 4 wherein one of the at least two double stranded polynucleotides provided in step i) is attached to a solid support.

10. Method of claim 1 to 4, wherein the double stranded polynucleotide is assembled by an automated process or a semi-automated process.

11. Method of claim 1, 3, and 4, wherein the at least one single stranded oligonucleotide provided in step i) of claim 1 and the at least one single stranded oligonucleotide provided in step ii) of claim 3 and 4 is derives from a single stranded tag library extracted from at least one biological source.

12. Method of claim 1, 3, and 4, wherein the at least one single stranded oligonucleotide provided in step i) of claim 1 and the at least one single stranded oligonucleotide provided in step ii) of claim 3 and 4 derives from a single stranded tag library extracted from at least one synthetic oligo/poly-nucleotide.

13. Method of claim 1, 3, and 4, wherein the three-dimensional structure of the resulting molecule in step iii) comprises a double-helix structure.

14. Method of claim 1, 3 and 4 wherein the at least one single stranded oligonucleotide provided in step i) of claim 1 and the at least one single stranded oligonucleotide provided in step ii) of claim 3 and 4 derives from a library comprising single stranded oligonucleotides comprising all possible sequence permutations of said single stranded oligonucleotide or any fraction of all possible sequence permutations of said single stranded oligonucleotide, such as at least 90% of all possible sequence permutations of said single stranded oligonucleotide.

15. Method of claim 1 wherein the at least three single stranded oligonucleotides with complementary nucleotide sequence parts provided step i) all have the same length.

16. Method of claim 1, 3, and 4, wherein the at least one single stranded polynucleotide provided in step i) of claim 1 and step ii) of claim 3 and 4, is between 1 and 30 bases long.

17. Method of claim 4 wherein at least one of the two double stranded polynucleotides provided in step i) is derived from a double stranded polynucleotide library extracted from at least one biological source.

18. Method of claim 4 wherein at least one of the two double stranded polynucleotides provided in step i) is derived from a synthetic double stranded polynucleotide library.

19. Method of claim 1, 3, and 4, wherein the at least one single stranded oligonucleotide provided in step i) of claim 1 and the at least one single stranded oligonucleotide provided in step ii) of claim 3 and 4 is at least one base longer than the combined length of its two complementary zip codes; providing a gap in one strand of the resulting polynucleotide assembly which can subsequently be closed by a DNA polymerase, or other method known in the art.
